(19) United States
(12) Patent Application Publication
(10) Pub. No.: US 2005/0143718 A1
(43) Pub. Date: Jun. 30, 2005

(54) Method for surgical treatment of a patient's eye by means of a laser

(75) Inventor: Christian Rathjen, Bremen (DE)

Correspondence Address: OLIFF & BERRIDGE, PLC, P.O. BOX 19928, ALEXANDRIA, VA, 22320, US

(73) Assignee: SIE AG SURGICAL INSTRUMENT ENGINEERING, Port (CH)

(21) Appl. No.: 11/000,883

(22) Filed: Dec. 2, 2004

Related U.S. Application Data
(63) Continuation of application No. 10/243,578, filed on Sep. 13, 2002, now Pat. No. 6,884,965.
Application No. 10/243,578 is a continuation-in-part of application No. 09/889,843, filed on Jul. 22, 2002.
(60) Provisional application No. 60/117,144, filed on Jan. 25, 1999.

(51) Int. Cl. A61B 18/20

(52) U.S. Cl. 606/5

(57) ABSTRACT

A method is provided for surgical treatment of a patient's eye (1) by means of a laser, producing ultra-short laser pulses, and a transparent reference body (3), disposed been a laser applicator (4) and the eye (1). A transparent foil (5) is disposed between the reference body (3) and the eye (1). When the reference body (3) is applied onto the eye (1), the foil (5) protects the eye (1) from direct contact by the reference body (3). The laser is activated and the laser pulses are projected from the laser applicator (4) through the reference body (3) and the foil (5) to dissolve tissue of the eye (1). By moving the focal point of the pulsed laser beam into the foil (5), open cuts can be made in the eye (1), without damaging the reference body (3).

### FIELD OF THE INVENTION

The present invention relates to a method for surgical treatment of a patient's eye by means of a laser. Specifically, the present invention relates to a method for surgical treatment of a patients eye by means of a laser, producing ultra-short laser pulses, and a transparent reference body, disposed between a laser applicator and the eye.

### BACKGROUND OF THE INVENTION

Laser technology has been used in ophthalmic surgery for many years. Significant progress in laser technology has made ft possible to also use laser technology for cutting a layer of the exterior surface of the cornea, a procedure that used to be performed before by means of a microkeratome using a mechanical blade. For cutting tissue inside the cornea, laser producing ultra-short laser pulses are used, for example pico second lasers producing pulse widths of 1 ps to 10 ps (1 ps=10⁻¹² s) or femto second lasers producing pulse widths of typically 1 fs to 1000 fs (1 fs=10⁻¹⁵ s). The required high intensities to dissolve tissue are achieved by focusing strongly the laser beam to a few microns. For cutting, it is necessary to place precisely pulses next to each other. For ophthalmic laser surgery to achieve sufficient precision, there needs to be a precise and stable coupling of the laser applicator to the operative area of the patients eye.

In U.S. Pat. No. 6,730,074, a system is described for accurately guiding a laser focal point along a predetermined path within the stroma of a cornea. The system includes a contact lens used as a reference body for the laser applicator. The contact lens is mounted in a suction ring and pressed gently against the exterior surface of the cornea. Applying a vacuum or a partial vacuum to the suction ring, the contact lens is held against the cornea. The laser applicator is moved relative to the contact lens. For example, the suction ring is fixed to the laser applicator.

In U.S. Pub. No. 2002/0103481, described is a stabilization and applanation device for reconfiguring the cornea of an eye for opthalmic laser surgery. The device includes an applanation lens used as a reference body for the laser applicator. A cone including the lens is connected to the laser applicator. A suction ring is attached to the eye and the cone is coupled to the suction ring. Thereby, the laser applicator is positioned in a defined relationship with the surface of the patient's eye applanated by the lens.

The reference bodies used in the prior art are in direct contact with the cornea of the patient's eye. Consequently, there is a danger of transferring infectious material, such as viruses, from one patient to another patient, if the same reference bodies are re-used without proper sterilization. Harm to the patient's eye may also be caused by abrasion, resulting from sliding movements, or by exposure to materials that are non-biocompatible. Using only disposable devices for contacting directly the patient's eye, as proposed in U.S. Pub. No. 2002/0103481, would help to prevent some of these problems. However, the reference bodies are precision devices and disposing thereof after one-time use causes a significant financial cost, to be covered by the patient or his medical insurance.

There are other risks and dangers to the patient's eye from having reference bodies in direct contact with the patient's eye, which cannot be prevented by limiting usage of reference bodies to one-time use. For example, when the reference body is in direct contact with the patient's eye, there is a risk that the patient's eye is being injured by sharp edges of a broken reference body. Injuries to the patients eye may also result from decomposition products of the reference body or deformations of the reference body, produced when the laser focal point is moved accidentally or deliberately into the area of the reference body.

### SUMMARY OF THE INVENTION

It is an object of this invention to provide an improved method for surgical treatment of a patient's eye by means of a laser, producing ultra-short laser pulses, and a transparent reference body, disposed between a laser applicator and the eye. In particular, it is an object of the present invention to provide a method for ophthalmic laser surgery, using ultra-short laser pulses and a transparent reference body, disposed between a laser applicator and the eye, without the disadvantages of the prior art. Particularly, the improved method for ophthalmic laser surgery should prevent the transfer from patient to patient of infectious material attached to reference bodies, without having to limit usage of a reference body to one-time. Moreover, the improved method for ophthalmic laser surgery should prevent injuries to the eye from broken or deformed reference bodies.

According to the present invention, in a method for surgical treatment of a patient's eye by means of a laser, producing ultra-short laser pulses, and a transparent reference body, disposed between a laser applicator and the eye, the above-mentioned objects are particularly achieved in that a transparent foil is disposed between the reference body and the eye. For example, the laser is a femto second laser or a pico second laser. Preferably, the foil is disposable. When the reference body is applied onto the eye, the foil is protecting the eye from direct contact by the reference body. In alternative embodiments, the reference body is coupled removably to the laser applicator or the reference body is fixed permanently to the laser applicator. The laser is activated and the ultra-short laser pulses are projected from the laser applicator through the reference body and the foil to dissolve tissue of the eye. In addition to protecting the eye from direct contact with the re-usable reference body, disposing a foil between the reference body and the eye has the advantage that the a focal point of the laser cannot only be adjusted to positions within cornea tissue of the eye, but also to positions within the foil to make open cuts in the cornea, without damaging the reference body. Moreover, disposing a foil between the reference body and the eye has the advantage that this makes it possible to choose freely the material used for the reference body.

In a preferred embodiment, disposing the foil between the reference body and the eye comprises attaching a suction ring to the eye around an operative area, the suction ring having attached thereon the foil such that the foil covers the operative area. Thereafter, the reference body is coupled removably to the suction ring such that the foil is interposed between the reference body and the eye.

In another embodiment, disposing the foil between the reference body and the eye comprises attaching the foil to a suction ring, attaching the suction ring to the eye around an operative area, and removably coupling the reference body to the suction ring such that the foil is interposed between the reference body and the eye.

In yet another embodiment, disposing the foil between the reference body and the eye comprises attaching a suction ring to the eye around an operative area, placing the foil into the suction ring to cover the operative area, and removably coupling the reference body to the suction ring such that the foil is interposed between the reference body and the eye.

In a further embodiment, disposing the foil between the reference body and the eye comprises placing the foil directly onto an operative area of the eye, attaching a suction ring to the eye to thereby stabilize the position of the foil relative to the operative area, and attaching the reference body to the suction ring such that the foil is interposed between the reference body and the eye.

In another embodiment, disposing the foil between the reference body and the eye comprises attaching the foil to the reference body prior to applying the reference body onto the eye.

In another alternative embodiment, disposing the foil between the reference body and the eye comprises attaching the foil to the laser applicator such that the part of the reference body to be applied onto the eye is covered by the foil, prior to applying the reference body onto the eye.

In various alternative or complementing embodiments, the method further comprises selecting the foil with a specified thickness to determine an operative depth, selecting the foil with a specified area of non-transparency to thereby limit an operative area of the eye, selecting the foil made of medical grade plastic, selecting the foil with a refraction index corresponding to the refraction index of the tissue of the eye and/or the refraction index of the reference body, selecting the foil with perforations to thereby exchange fluids between an interior surface of the foil contacting the eye and an exterior surface of the foil contacting the reference body, selecting the foil with surface structures on an interior surface of the foil contacting the eye to thereby conduct fluids from and to an exterior surface of the eye being contacted by the foil, and/or selecting the foil with elastic coating on an interior surface of the foil contacting the eye to thereby even out uneven areas of an exterior surface of the eye being contacted by the foil.

### BRIEF DESCRIPTION OF THE DRAWINGS

The present invention will be explained in more detail, by way of example, with reference to the drawings in which:

FIG. 1a shows a schematic cross section of the cornea of a patient's eye wherein a suction ring, having a transparent foil fixed thereon, is attached to the eye.

FIG. 1b shows the schematic cross section of the cornea of FIG. 1a wherein a reference body, fixed to a laser applicator, is coupled to the suction ring such that the transparent foil is interposed between the reference body and the eye.

FIG. 2a shows a schematic cross section of the cornea of a patient's eye wherein a suction ring, having a transparent foil placed therein, is attached to the eye.

FIG. 2b shows the schematic cross section of the cornea of FIG. 2a wherein a reference body, fixed to a laser applicator, is coupled to the suction ring such that the transparent oil is interposed between the reference body and the eye.

FIG. 3a shows a schematic cross section of the cornea of a patient's eye wherein a transparent foil is placed directly onto the eye and a suction ring, attached to the eye, stabilizes the position of the foil relative to the eye.

FIG. 3b shows the schematic cross section of the cornea of FIG. 3a wherein a reference body, fixed to a laser applicator, is coupled to the suction ring such that the transparent foil is interposed between the reference body and the eye.

FIG. 4a shows a schematic cross section of the cornea of a patient's eye wherein a suction ring, having a transparent foil placed therein, is attached to the eye, and wherein a reference body is held in the suction ring such that the transparent foil is interposed between the reference body and the eye

FIG. 4b shows the schematic cross section of the cornea of FIG. 4a wherein a laser applicator is coupled to the suction ring and to the reference body.

FIG. 5a shows a schematic cross section of the cornea of a patients eye, having a suction ring attached thereto, and a laser applicator with a reference body covered by a transparent foil attached to the laser applicator.

FIG. 5b shows the schematic cross section of the cornea of FIG. 5a wherein the reference body and the laser applicator are coupled to the suction ring such that the transparent foil is interposed between the reference body and the eye.

FIG. 6a shows a schematic cross section of the cornea of a patient's eye, having a suction ring attached thereto, and a laser applicator, having a reference body with a transparent foil fixed thereon.

FIG. 6b shows the schematic cross section of the cornea of FIG. 6a wherein the reference body and the laser applicator are coupled to the suction ring such that the transparent foil is interposed between the reference body and the eye.

FIG. 7a shows a schematic cross section of the cornea of a patient's eye and of a suction ring, having a transparent foil fixed thereon, coupled to the laser applicator.

FIG. 7b shows the schematic cross section of the cornea of FIG. 7a wherein the suction ring, coupled to the laser applicator, is attached to the eye such that the transparent foil is interposed between the reference body and the eye.

FIG. 8 shows a schematic cross section of the cornea of a patient's eye, having a transparent foil placed thereon, wherein different positions of the focal point of a pulsed laser beam, projected through a reference body and the foil, are illustrated.

### DETAILED DESCRIPTION OF THE PREFERRED EMBODIMENTS

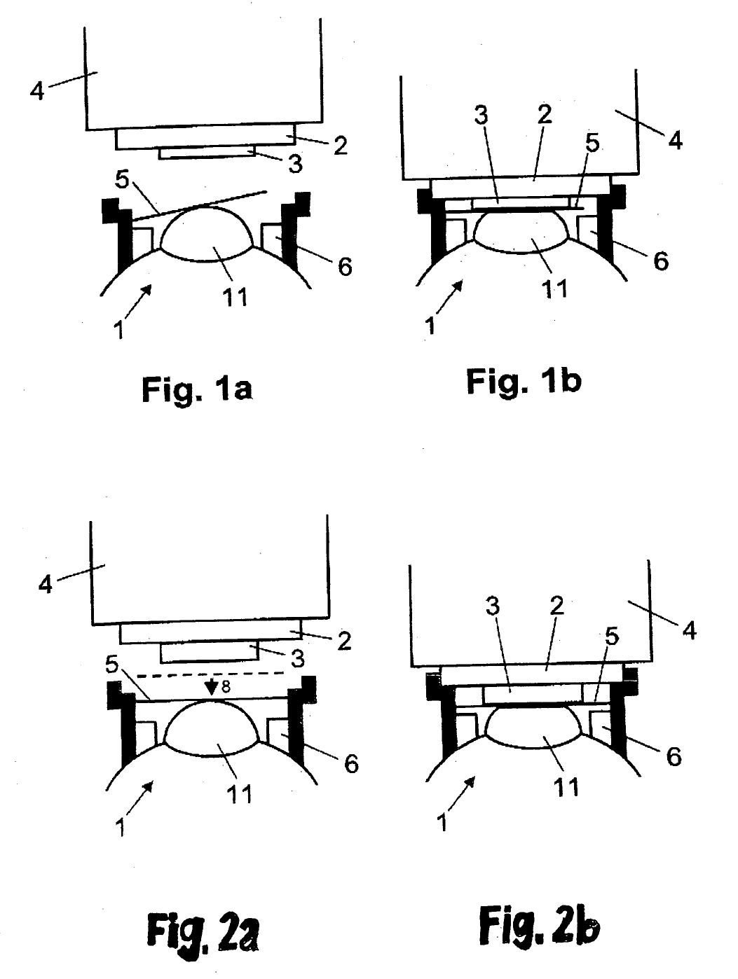

In FIGS. 1a, 1b, 2a, 2b, 3a, 3b, 4a, 4b, 5a, 5b, 6a, 6b, 7a, 7b, and 8, the reference numeral 1 refers to a patients eye and the reference numeral 11 refers to the cornea of the eye 1.

In FIGS. 1a, 1b, 2a, 2b, 3a, 3b, 4a, 4b, 5a, 5b, 6a, 6b, 7a, 7b, and 8 the reference numeral 4 refers to a laser applicator. Preferably, the laser applicator 4 is configured to project pulsed and focused laser beams having ultra-short laser pulses. For example, the laser applicator 4 is configured to project pulse widths of 1 ps to 10 ps (pico second laser) or pulse widths of typically 1 fs to 1000 fs (femto second laser). The laser is preferably integrated in the laser applicator 4. However, it is also possible to have the laser external to the laser applicator 4, coupled through optical fibers. As is illustrated in FIGS. 1a, 1b, 2a, 2b, 3a, 3b, 4a, 4b, 5a, 5b, 6a, 6b, 7a, and 7b, the laser applicator 4 includes an applicator end piece 2.

As is illustrated in FIGS. 1a, 1b, 2a, 2b, 3a, 3b, 5a, 5b, 6a, 6b, 7a, 7b, and 8, a reference body 3 is attached to the applicator end piece 2, i.e. to the laser applicator 4. The reference body 3 is either permanently fixed to the laser applicator 4 or coupled removably to the laser applicator 4, for example by means of a snap or screw mechanism. The reference body 3 is transparent and preferably made from clear (optical) glass. The reference body 3 may have a cylindrical shape or the shape of a contact lens, for example.

In FIGS. 1a, 1b, 2a, 2b, 3a, 3b, 4a, 4b, 5a, 5b, 6a, 6b, 7b, and 8, the reference numeral 6 refers to a suction ring, which is held in place on the patient's eye 1 by means of a vacuum or a partial vacuum generated by a vacuum pump (not illustrated). The suction ring 6 and the laser applicator 4 are configured for removably interconnecting with each other, to thereby stabilize the position of the pulsed laser beam relative to the patent's eye.

In FIGS. 1a, 1b, 2a, 2b, 3a, 3b, 4a, 4b, 5a, 5b, 6a, 6b, 7a, 7b, and 8, the reference numeral 5 refers to a foil, which is transparent at least in some areas so that a pulsed laser beam can be projected through the foil 5 in those areas. The foil 5 is flexible and has a thickness of less than one millimeter. Preferably, the foil 5 is provided in different defined thickness so that a specific foil 5, interposed between reference body 3 and the eye, can be selected to adjust the position of the focal point of the pulsed laser beam to thereby determine the operative depth. Preferably, the foil 5 is made from medical grade plastic and has a refraction index corresponding to the refraction index of the tissue of the eye 1 and/or the refraction index of the reference body 3. In an embodiment, the foil is provided with perforations to make possible the exchange of fluids between the interior surface of the foil 5 contacting the eye 1 and the exterior surface of the foil 5 contacting the reference body 3. In a further embodiment, the foil 5 is provided with surface structures on the interior surface to make it possible to conduct fluids from and to the exterior surface of the eye 1 being contacted by the foil 5. In a further embodiment, the foil 5 is provided with an elastic coating on the interior surface of the foil 5 contacting the eye 1 to make it possible to even out uneven areas, for example scars, of the exterior surface of the eye 1.

In the following paragraphs, different embodiments of disposing the sterile foil 5 between the reference body 3 and an operative area of the eye 1 are described with reference to FIGS. 1a, 1b, 2a, 2b, 3a, 3b, 4a, 4b, 5a, 5b, 6a, 6b, 7a, and 7b. In the examples illustrated, the operative area is part of the cornea 11.

In the preferred embodiment illustrated in FIG. 1a, first, a suction ring 6, having the transparent foil 5 attached thereon, is placed around the operative area of the eye 1 such that the foil 5 covers the operative area. The foil 5 is either fixed permanently to the suction ring 3 (e.g. by the manufacturer) or is attached removably to the suction ring 6, for example through adhesion or by fastening means such as a clamping mechanism. Thereafter, the suction ring 6 is attached to the eye 1. As is illustrated in FIG. 1b, the reference body 3 is applied onto the eye 1 in that the laser applicator 4, with the reference body 3 attached thereon, is coupled to the suction ring 6. Through coupling the laser applicator 4 to the suction ring 6, the foil 5 is interposed between the reference body 3 and the eye 1.

In the embodiment illustrated in FIG. 2a, first, the suction ring 6, is placed around the operative area of the eye 1 and attached to the eye 1. Then, as is indicated schematically by arrow 8, the foil 5 is placed into the opening of the suction ring 6 such that the foil 5 covers the operative area. Thereafter, as illustrated in FIG. 2b, the laser applicator 4, with the reference body 3 attached thereon, is coupled to the suction ring 6. Thereby, the foil 5 is interposed between the reference body 3 and the eye 1.

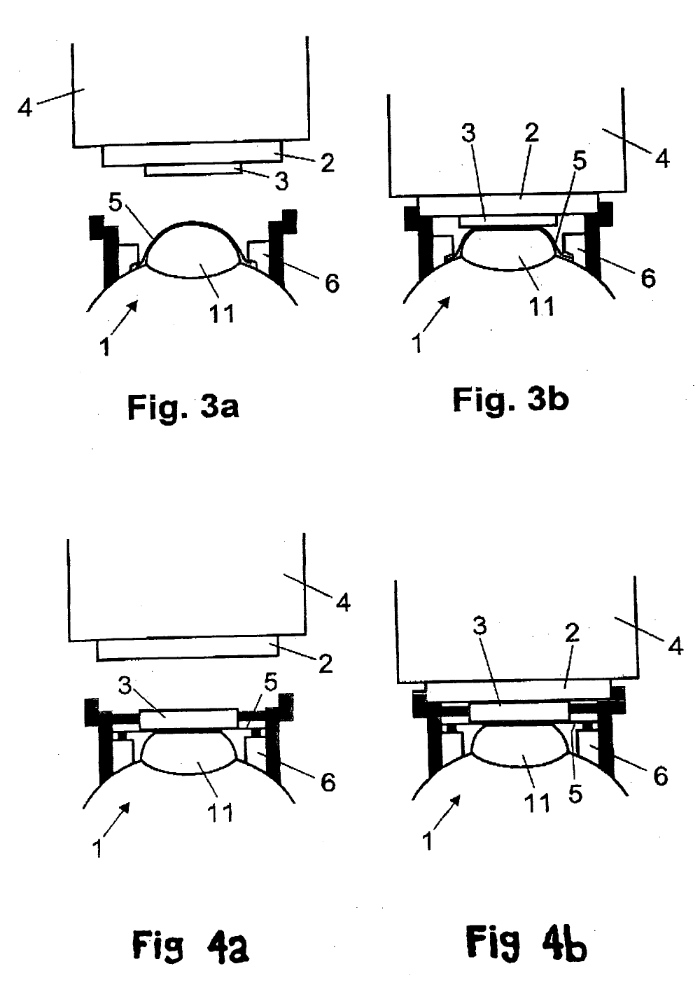

In the embodiment illustrated in FIG. 3a, first, the transparent foil 5 is placed directly onto the eye 1 to cover the operative area. Thereafter, the suction ring 6 is placed onto the eye 1 such that foil 5 is stabilized relative to the operative area by the suction ring 6. Finally, as illustrated in FIG. 3b, the laser applicator 4, with the reference body 3 attached thereon, is coupled to the suction ring 6. Thereby, the foil 5 is interposed between the reference body 3 and the eye 1.

In the embodiment illustrated in FIG. 4a, a suction ring 6, having the transparent foil 5 pod therein and the reference body 3 attached thereto, is placed around the operative area and attached to the eye 1. The foil 5 is placed into the suction ring 6, before the reference body 3 is attached to me suction ring 6. The foil 3 is either fixed permanently to the suction ring 6, for example by the manufacturer, or placed removably into the suction ring 6. The reference body 3 is coupled removably to the suction ring 6, for example by means of a snap or screw mechanism. When attaching the suction ring 6 to the eye 1, in this embodiment, the foil 5 is interposed between the reference body 3 and the eye 1. Finally, as illustrated in FIG. 4b, the laser applicator 4, is coupled to the suction ring 6 and to the reference body 3. In an alternative embodiment, the reference body 3 is fixed permanently to the suction ring 6 or manufactured with the suction ring 6 as one piece.

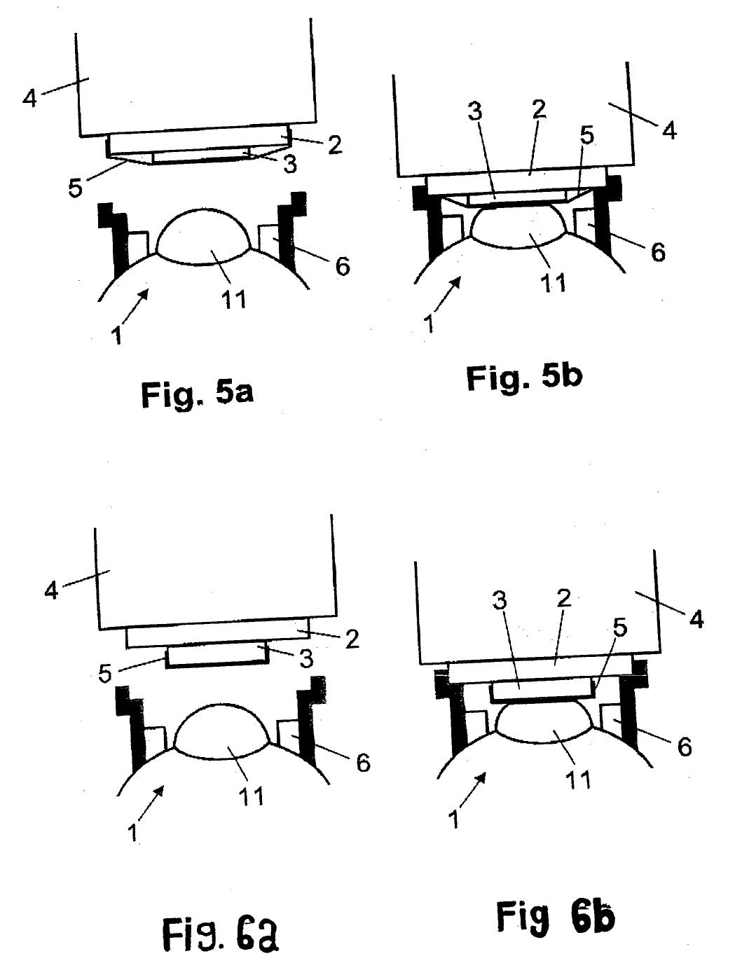

In the embodiment illustrated in FIG. 5a, the foil 5 is attached to the laser applicator 4 (or to the applicator end piece 2, respectively) such that the foil 5 covers the part of the reference body 3 to be applied onto the eye 1. The foil 5 is attached removably to the laser applicator 4, for example through adhesion, vacuum, or by fastening means such as a clamping mechanism. After having placed the suction ring 6 around the operative area and attached to the eye 1, the laser applicator 4, with the covered reference body 3 attached thereto, is coupled to the suction ring 6. Thereby, the foil 5 is interposed between the reference body 3 and the eye 1, as illustrated in FIG. 5b.

In the embodiment illustrated in FIG. 6a, the foil 5 is attached to the reference body 3 attached to the laser applicator 4 (or to the applicator end piece 2, respectively) such that the foil 5 covers the part of the reference body 3 to be applied onto the eye 1. The foil 5 is attached removably to the reference body 3 through adhesion or by fastening means such as a clamping mechanism. After having placed the suction ring 6 around the operative area and attached to the eye 1, the laser applicator 4, with the covered reference body 3 attached thereto, is coupled to the suction ring 6. Thereby, the foil 5 is interposed between the reference body 3 and the eye 1, as illustrated in FIG. 6b.

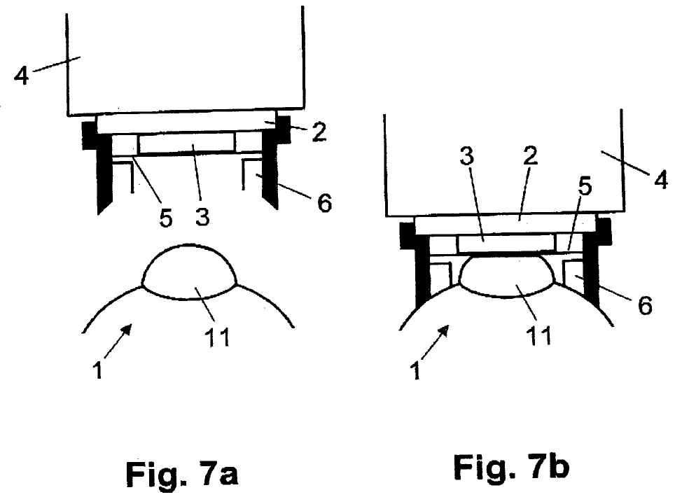

As is illustrated in 7a, in a preferred embodiment, the suction ring 6 and the foil 5 are coupled removably to the laser applicator 4 prior to attaching the suction ring 6 to the eye 1, as shown in FIG. 7b. Attaching the suction ring 6 to the eye 1 after having coupled the suction ring 6 to the laser applicator 4 and the reference body 3 is applicable also to the embodiments illustrated in FIGS. 1a, 1b, 2a, 2b, 3a, 3b, 4a, 4b, 5a, 5b, 6a, and 6b.

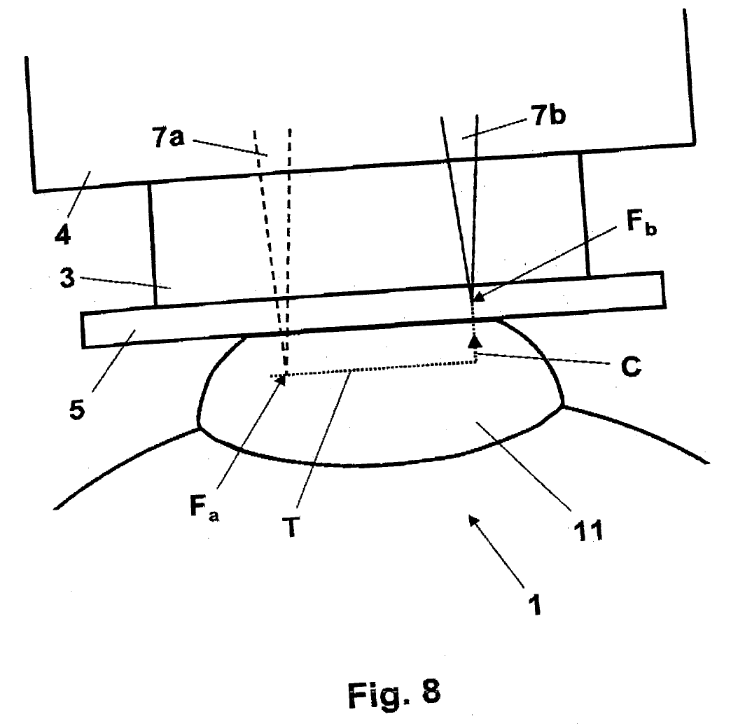

When the laser is activated for surgical treatment of the patient's eye 1, the laser pulses are projected from the laser applicator 4 through the reference body 3 and the foil 5. For example, if the focal point Fₐ of the pulsed laser beam is located at a position within the cornea 11, as illustrated in FIG. 8, the laser pulses dissolve tissue of the cornea 11. To make a cut within the cornea, the focal point of the pulsed laser beam is moved along the line T, for example. To make an open cut, the focal point F_{b} of the pulsed laser beam is moved along the line C into the foil 5, thereby not damaging the reference body 3.

It must be pointed out that the configurations of the suction ring 6, the foil 5, the laser applicator 4, and the reference body 3 are illustrated schematically only and that alternative configurations of these components are possible without deviating from the scope of the invention. One skilled in the art will also understand that the steps illustrated and described for disposing the transparent foil 5 between the reference body 3 and the eye 1 can be executed in alternative sequences and combinations. For example, removably coupling the reference body 3 to the suction ring 6, as described in the context of FIG. 4a, may also be combined with the application of the suction ring 6 and the transparent foil 5 as described in the context of FIGS. 1a, 2a, and 3a.

1. A method for surgical treatment of a patient's eye by means of a laser, producing ultra-short laser pulses, and a transparent reference body disposed between a laser applicator and the eye, the method comprising:
disposing a transparent foil between the reference body and the eye;
applying the reference body onto the eye, the foil protecting the eye from direct contact by the reference body; and
activating the laser and projecting the laser pulses from the laser applicator through the reference body and the foil to dissolve tissue of the eye.

2. The method according to claim 1, wherein disposing the foil between the reference body and the eye comprises:
attaching a suction ring to the eye around an operative area, the suction ring having attached thereon the foil such that the foil covers the operative area; and
removably coupling the reference body to the suction ring such that the foil is interposed been the reference body and the eye.

3. The method according to claim 1, wherein disposing the foil between the reference body and the eye comprises:
attaching the foil to a suction ring;
attaching the suction ring to the eye around an operative area; and
removably coupling the reference body to the suction ring such that the foil is interposed between the reference body and the eye.

4. The method according to claim 1, wherein disposing the foil between the reference body and the eye comprises:
attaching a suction ring to the eye around an operative area;
placing the foil into the suction ring to cover the operative area; and
removably coupling the reference body to the suction ring such that the foil is interposed between the reference body and the eye.

5. The method according to claim 1, wherein disposing the foil between the reference body and the eye comprises:
placing the foil directly onto an operative area of the eye;
attaching a suction ring to the eye to thereby stabilize the position of the foil relative to the operative area; and
attaching the reference body to the suction ring such that the foil is interposed between the reference body and the eye.

6. The method according to claim 1, wherein disposing the foil between the reference body and the eye comprises:
attaching the foil to the reference body prior to applying the reference body onto the eye.

7. The method according to claim 1, wherein disposing the foil between the reference body and the eye comprises:
attaching the foil to the laser applicator, prior to applying the reference body onto the eye, such that the foil covers the part of the reference body to be applied onto the eye.

8. The method according to claim 1, wherein disposing the foil between the reference body and the eye comprises removably coupling a suction ring to the laser applicator prior to applying the reference body onto the eye, the suction ring having attached thereon the foil such that the foil covers the part of the reference body to be applied onto the eye; and wherein applying the reference body onto the eye comprises attaching the suction ring with the laser applicator coupled thereto to the eye around an operative area such that the foil is interposed between the reference body and the eye.

9. The method according to claim 1, further comprising adjusting a focal point of the laser to a position within cornea tissue of the eye.

10. The method according to claim 1, further comprising adjusting a focal point of the laser from a position within cornea tissue of the eye to a position within the foil.

11. The method according to claim 1, further comprising selecting the foil with a specified thickness to determine an operative depth.

12. The method according to claim 1, further comprising selecting the foil with a specified area of non-transparency to thereby limit an operative area of the eye.

13. The method according to claim 1, further comprising selecting the foil made of medical grade plastic.

14. The method according to claim 1, further comprising selecting the foil with a refraction index corresponding to the refraction index of the tissue of the eye and/or the refraction index of the reference body.

15. The method according to claim 1, further comprising selecting the foil with perforations to thereby exchange fluids between an interior surface of the foil contacting the eye and an exterior surface of the foil contacting the reference body.

16. The method according to claim 1, further comprising selecting the foil with surface structures on an interior surface of the foil contacting the eye to thereby conduct fluids from and to an exterior surface of the eye being contacted by the foil.

17. The method according to claim 1, further comprising selecting the foil with elastic coating on an interior surface of the foil contacting the eye to thereby even out uneven areas of an exterior surface of the eye being contacted by the foil.

18. The method according to claim 1, wherein the reference body is coupled removably to the laser.

19. The method according to claim 1, wherein the reference body is fixed permanently to the lasers.

20. The method according to claim 1, wherein the laser is a femto second laser or a pico second laser.

21. The method according to claim 1, wherein the foil is disposable.
